(19) United States
(12) Patent Application Publication
(10) Pub. No.: US 2025/0387034 A1
(43) Pub. Date: Dec. 25, 2025

(54) System and Method for Real-Time Heart Rate Communication Using Haptic and Visual Feedback

(71) Applicant: Enrique Artinano, Zapopan (MX)

(72) Inventor: Enrique Artinano, Zapopan (MX)

(21) Appl. No.: 18/748,150

(22) Filed: Jun. 20, 2024

(51) Int. Cl. A61B 5/024; A61B 5/00; A61B 5/11

(52) U.S. Cl. CPC A61B 5/024; A61B 5/112; A61B 5/681; A61B 5/7415; A61B 5/742; A61B 5/7455; A61B 2560/0209; A61B 2560/0214; A61B 2560/0276; A61B 2560/045; A61B 2560/0462; A61B 2562/0219; A61B 2562/0271

(57) ABSTRACT

A system for real-time heart rate communication comprises a handheld device configured to be held by a user, containing a control unit, a communication module, a feedback mechanism, and a power source. The communication module receives heart rate data from a first user device, which processes the data and generates control instructions. The first user device receives physiological data including heart rate data from a second user device which may be either a smartwatch or a second smartphone. The feedback mechanism, including a vibration motor and RGB LEDs, provides synchronized haptic and visual feedback based on the received heart rate data. The system enables users to experience heart rate data through sensory feedback, enhancing emotional connectivity and situational awareness.

### FIELD OF INVENTION

The present invention relates generally to the field of physiological data communication and feedback systems. Specifically, it involves a system and method for real-time heart rate communication between users through a dedicated handheld device that provides synchronized haptic and visual feedback.

### BACKGROUND

The rapid evolution of portable user terminals and multimedia communication devices has enabled unprecedented ways of sharing data and experiences. Despite these advancements, conveying the intimate and personal nuances of a user's physiological state, such as heart rate, through digital means remains a challenge. Existing solutions often rely heavily on audiovisual methods, which are limited in their capacity to convey the full spectrum of human emotions and sensations. Consequently, there is a significant need for innovative methods that provide a more immersive and tactile experience.

In recent years, wearable technology has seen significant growth, with devices such as smartwatches and fitness trackers becoming ubiquitous. These devices often include sensors to monitor physiological parameters like heart rate, steps taken, and sleep patterns, providing valuable health insights to the user. However, the primary focus of these devices is on health and fitness tracking, with their feedback mechanisms typically limited to visual displays or simple haptic alerts. This narrow scope fails to leverage the full potential of haptic and visual feedback to create a deeper, more personal connection between users.

Further advancements in the Internet of Things (IoT) have enabled devices to communicate more seamlessly, opening up new possibilities for remote interactions. Despite this, the application of IoT technology in personal, emotional communication remains underdeveloped. While there are systems that allow users to share data and control devices remotely, these interactions are often transactional rather than experiential. The challenge lies in creating a device that not only transmits data but also recreates the sender's physiological state in a manner that can be felt and experienced by the receiver.

KR20200009636A addresses part of this challenge by introducing a system that simulates a user's heartbeat through communication between user devices. This system allows for the transmission of heart rate data between devices, enabling one user to feel the heartbeat of another through vibrations. However, this approach is constrained by its reliance on the users' phones or wearables, lacking a dedicated device designed specifically for this purpose. The absence of a standalone, handheld device limits the potential for a more immersive and specialized user experience that a dedicated device could provide.

CN112439119A presents a real-time feedback system for respiratory regulation and stress relief, using a handheld device to provide feedback through touch, vibrations, and light. While this system effectively addresses stress reduction and breathing synchronization, it is primarily focused on respiratory patterns rather than heart rate communication. The device's design and functionality are geared towards guiding breathing exercises and meditation, making it less suitable for applications requiring real-time heart rate data transmission and simulation.

The limitations of these existing systems, including their reliance on multipurpose devices and focus on different physiological parameters, highlight the need for a specialized solution. A dedicated handheld device designed specifically to transmit and simulate heart rate sensations through synchronized vibrations and RGB LED lights could offer a more direct and emotionally engaging experience. This would provide a unique way for individuals to share and feel heart rate patterns in real-time, enhancing emotional connection and offering situational awareness that current solutions do not fully address.

It is within this context that the present invention is provided.

### SUMMARY

The present invention provides a system for real-time heart rate communication, comprising a handheld device containing a control unit, a communication module, a feedback mechanism, and a power source. The control unit within the handheld device interprets control instructions and actuates the feedback mechanism to provide synchronized sensory feedback, such as haptic and visual cues, based on received heart rate data. The communication module receives these control instructions via a communication protocol from a first user device, which processes the heart rate data and generates control instructions for the handheld device. The first user device receives heart rate data from a second user device, which could be a smartwatch or another smartphone.

In some embodiments, the communication module within the handheld device is a Bluetooth Low Energy (BLE) module, facilitating efficient and low-power wireless communication.

In further embodiments, the feedback mechanism includes a vibration motor that provides haptic feedback based on the received physiological data.

In yet further embodiments, the feedback mechanism also includes a plurality of RGB LEDs that offer visual feedback. The control unit can control these LEDs to emit light synchronized with the heart rate data.

In some embodiments, the system is powered by a rechargeable battery housed within the device, ensuring portability and ease of use.

In further embodiments, the system includes a battery management system connected to the power source to manage the charging and discharging processes effectively.

In yet further embodiments, the feedback mechanism incorporates an audio output device that provides auditory feedback based on the received physiological data.

In some embodiments, the first user device receives physiological data from a smartwatch, enhancing compatibility with common wearable technology.

In further embodiments, the first user device can also receive physiological data from another smartphone, increasing the flexibility of the system.

In yet further embodiments, the system includes a secure pairing process for establishing a reliable connection between the communication module and the first user device, ensuring data integrity and security.

In some embodiments, the system features a user interface on the first user device application, allowing users to customize feedback settings on the handheld device.

In further embodiments, the customizable feedback settings include options for adjusting vibration intensity levels and RGB LED color patterns.

In yet further embodiments, the control unit is programmed to log physiological data and feedback patterns over time, enabling analysis and tracking of the user's physiological responses.

In some embodiments, the system includes a sensor that detects the temperature within the housing and adjusts the feedback mechanism to prevent overheating, thereby protecting the device and the user.

In further embodiments, the temperature sensor triggers an automatic shutdown of the feedback mechanism if the temperature exceeds a predefined threshold, ensuring safe operation.

In yet further embodiments, the system features a charging port and indicator LEDs that display the battery level and charging status, providing users with clear information on the device's power state.

In some embodiments, the housing of the handheld device includes a customizable exterior cover that users can replace or modify, offering personalization options.

In further embodiments, the system includes an integrated memory module for storing received physiological data and feedback patterns, facilitating data storage and retrieval.

In yet further embodiments, the control unit is configured for wireless firmware updates via the communication module, ensuring the device remains current with the latest software enhancements.

In some embodiments, the system includes a motion sensor that detects the handheld device's movement and adjusts the feedback mechanism accordingly, providing context-aware feedback.

In further embodiments, the physiological data processed by the system includes information on the user's activity type, and the feedback mechanism adjusts its responses based on this data.

In yet further embodiments, the system incorporates a GPS module for tracking the location of the handheld device, adding an additional layer of functionality.

### BRIEF DESCRIPTION OF THE DRAWINGS

Various embodiments of the invention are disclosed in the following detailed description and accompanying drawings.

FIG. 1 illustrates an example first embodiment of the handheld device being held in a user's hand, showing its communication with a smartphone receiving data from a different user's smartwatch.

FIG. 2 illustrates an example block diagram of the handheld device of the first embodiment, detailing its internal components and software modules.

FIG. 3 illustrates an example process flow diagram of the first embodiment representing the sequence of operations during the transmission of physiological data from a smartwatch to a smartphone and then to the handheld device, which emulates the physiological signals.

FIG. 4 illustrates an example second embodiment of the handheld device being held in a user's hand, showing its communication with a smartphone receiving data from a different user's smartphone, which in turn has received said data form their smartwatch.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### Detailed Description and Preferred Embodiment

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The term “housing” refers to the external casing or structure of the handheld device that houses its internal components. This housing can be made from various materials, such as plastic, metal, or composite materials, chosen for their durability, weight, and ergonomic properties. For example, in one implementation, the housing may be constructed from a lightweight, impact-resistant polymer with a soft-touch finish, designed to be comfortable to hold and visually appealing. The design of the housing may include ergonomic contours to enhance user comfort during prolonged use.

The term “control unit” refers to a compact integrated circuit designed to govern the operation of the handheld device. It may be as simple or as sophisticated as required by the specific implementation it is used in. It includes a processor, memory, and input/output peripherals. In some embodiments, the control unit may be capable of executing complex control algorithms while maintaining low energy consumption. The control unit's firmware can be programmed to handle data processing, control feedback mechanisms, and manage communication protocols, ensuring seamless operation of the device.

The term “communication module” refers to the component responsible for transmitting and receiving data between the handheld device and external devices, such as smartwatches or smartphones. This module typically utilizes wireless communication standards such as Bluetooth Low Energy (BLE). For instance, the communication module may be a Bluetooth 5.0 chip that supports long-range communication and enhanced data throughput. It enables the device to establish a secure connection with external devices, facilitating the transfer of physiological data in real-time. It may also use cloud architectures and standards for communications.

The term “feedback mechanism” encompasses all components that provide sensory feedback to the user, including haptic and visual elements. This mechanism may include a vibration motor and RGB LEDs. For example, the vibration motor can be a coin-type vibrator or a linear resonant actuator, capable of producing varying intensity vibrations to simulate the sensation of a heartbeat. The RGB LEDs may be controlled by the control unit to emit light in different colors and intensities, synchronized with the user's heart rate. This setup enhances the user's experience by providing both tactile and visual cues that reflect the physiological data.

The term “power source” refers to the component that supplies electrical power to the device's components. It is typically a rechargeable battery, such as a lithium-polymer (Li—Po) or lithium-ion (Li-ion) battery, known for their high energy density and long cycle life. For instance, the power source may be a 3.7V Li—Po battery with a capacity of 1000 mAh, capable of powering the device for several hours of continuous use. The power source is equipped with a battery management system (BMS) to monitor voltage, current, and temperature, ensuring safe and efficient charging and discharging.

The term “firmware” refers to the software programmed into the control unit to control the device's hardware and manage its operations. This software includes algorithms for processing physiological data, controlling the feedback mechanisms, and handling communication protocols. For example, the firmware may include routines to parse heart rate data received from the smartwatch, adjust the vibration intensity and LED colors based on real-time data, and manage the device's power consumption to maximize battery life.

The term “sensor” refers to any device or component that detects and measures physical parameters, such as temperature or motion, within the housing. In some embodiments, the sensor may be a temperature sensor, such as the LM35, which monitors the internal temperature of the device and triggers thermal management actions if the temperature exceeds a predefined threshold. This ensures the device operates within safe thermal limits, preventing overheating and ensuring consistent performance.

The term “secure pairing process” refers to the methods and protocols used to establish a trusted and secure connection between the handheld device and external devices. This process may involve techniques such as Just Works, Passkey Entry, or Numeric Comparison, as defined by Bluetooth standards. For instance, the pairing process may require users to confirm a numerical passkey displayed on both the handheld device and the external device, ensuring that the connection is secure and that unauthorized devices cannot access the data.

The term “user interface” refers to any software or hardware component that allows a user to interact with the handheld device and customize its settings. This includes, but is not limited to, graphical user interfaces (GUIs) on mobile applications or web-based dashboards. For example, the user interface may be implemented as a mobile application that allows users to adjust vibration intensity, LED color patterns, and other settings through a touchscreen interface. The application may also display real-time data and historical logs, providing users with insights into their physiological metrics.

### DESCRIPTION OF DRAWINGS

The present invention relates to a system and method for real-time heart rate communication using haptic and visual feedback. The invention addresses several shortcomings of existing technologies, which predominantly rely on audiovisual means to convey physiological data, thereby failing to fully engage users in a tactile and immersive manner. This invention provides a dedicated handheld device that integrates seamlessly with smartwatches and smartphones to deliver synchronized haptic and visual feedback based on the user's heart rate data.

Traditional systems, such as those disclosed in KR20200009636A, simulate a user's heartbeat through communication between user devices but are limited by their reliance on phones or wearable devices. These systems do not provide a dedicated, handheld solution specifically designed for this purpose, thus limiting the potential for a more immersive user experience. Furthermore, existing solutions like CN112439119A focus on respiratory regulation and stress relief using handheld devices that offer feedback through vibrations and light but are not tailored for heart rate communication. These devices are primarily designed for guiding breathing exercises and do not address the need for real-time heart rate data transmission and feedback.

The invention described herein overcomes these limitations by providing a dedicated handheld device that offers a more direct and engaging method for users to experience heart rate data. The device comprises a housing that encloses a control unit, a communication module, a feedback mechanism, and a power source. The communication module receives physiological data, such as heart rate, from external devices like smartwatches and smartphones. The control unit processes this data and controls the feedback mechanism to provide synchronized haptic and visual feedback, allowing users to feel and see the heart rate patterns in real-time.

The primary benefits of this invention include enhanced emotional connectivity and situational awareness. By providing a tactile and visual representation of physiological data, the device fosters a more profound sense of presence and connection between users. Additionally, the invention supports customizable feedback settings, secure data transmission, and the ability to log and analyze historical data, offering a comprehensive solution for real-time heart rate communication.

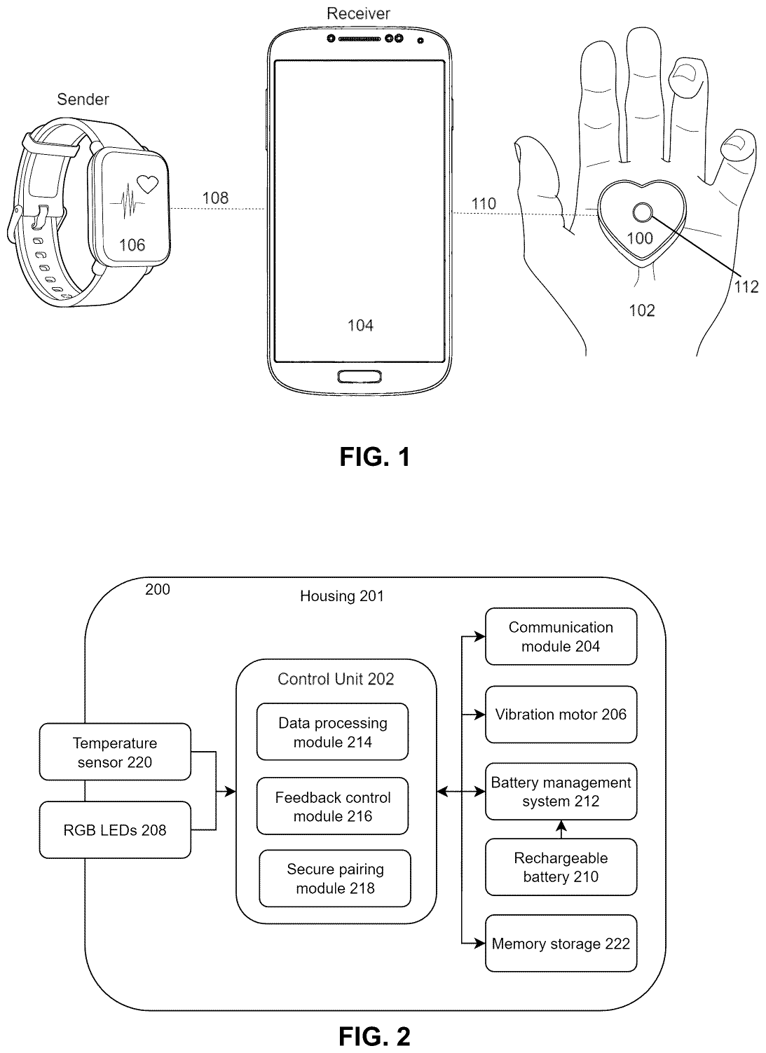

Referring now to the drawings, FIG. 1 illustrates an embodiment of the system for real-time heart rate communication, depicting the heart-shaped handheld device 100 in the user's hand 102. The device 100 is designed to provide synchronized haptic and visual feedback based on heart rate data received from an external source. The device 100 includes a housing configured to be ergonomically held, and within this housing is a control unit, a communication module, a feedback mechanism, and a power source.

The user's smartphone 104, which receives heart rate data from a different user's smartwatch 106, is shown in communication with the device 100. The smartwatch 106, worn by a second user, continuously monitors heart rate data using integrated sensors. This data is transmitted to the smartphone 104 via a Bluetooth Low Energy (BLE) connection 108. The smartphone 104 processes the heart rate data and transmits it to the handheld device 100 using another BLE connection 110.

The control unit within the device 100 processes the received heart rate data and controls the feedback mechanism. This feedback mechanism includes a vibration motor and a set of RGB LEDs 112. As shown in FIG. 1, the device 100 is vibrating in sync with the heartbeat data received from the smartwatch 106. The vibration motor produces haptic feedback that mimics the rhythm of the heartbeats, providing a tactile representation of the heart rate data.

Simultaneously, the RGB LEDs 112 emit light patterns corresponding to the heart rate data. The control unit adjusts the color and intensity of the LEDs to reflect different heart rate zones, such as resting, active, or stressed states. The LEDs can display colors like blue for resting, orange for stressed, and green for active, providing a visual cue to the user.

The smartphone application running on the user's smartphone 104 facilitates this entire process by securely pairing with the smartwatch 106 and the handheld device 100. The application allows the user to customize settings such as vibration intensity and LED color patterns. The application also logs the heart rate data and feedback patterns, enabling users to review their physiological data over time

In this embodiment, the handheld device 100 includes a rechargeable battery that powers the control unit, communication module, vibration motor, and RGB LEDs. The battery management system ensures safe and efficient charging and discharging, monitored by integrated sensors that detect temperature and prevent overheating.

FIG. 2 illustrates a block diagram of the handheld device 200, the elements disposed on the housing 201, and its internal components, as well as the software modules run by the control unit. The device 200 is designed to provide real-time heart rate communication through synchronized haptic and visual feedback.

The core of the device 200 is the control unit 202, which is responsible for processing the received physiological data and controlling the feedback mechanisms. The control unit 202 is connected to the communication module 204, which receives heart rate data from an external device such as a smartphone. The communication module 204 utilizes Bluetooth Low Energy (BLE) to establish a secure connection with the smartphone and receive the data.

The feedback mechanism of the device 200 comprises a vibration motor 206 and a set of RGB LEDs 208. The control unit 202 controls the vibration motor 206 to produce haptic feedback that synchronizes with the heart rate data. The RGB LEDs 208 are also controlled by the control unit 202 to emit visual feedback in various colors and intensities based on the heart rate data.

The device 200 is powered by a rechargeable battery 210, which supplies power to all the internal components. The battery 210 is managed by a battery management system 212 that ensures safe and efficient charging and discharging. The battery management system 212 monitors the battery's voltage, current, and temperature to prevent overheating and prolong battery life.

The control unit 202 runs several software modules to manage the device's operations. These include the data processing module 214, which processes the received heart rate data and prepares it for feedback. The feedback control module 216 manages the synchronization of the vibration motor 206 and RGB LEDs 208 with the processed heart rate data.

Additionally, the device 200 includes a secure pairing module 218, which facilitates the secure connection between the communication module 204 and external devices. This module implements encryption and authentication protocols to protect the data transmitted to and from the device 200.

The temperature sensor 220 within the device 200 monitors the internal temperature and communicates with the control unit 202 to prevent overheating. If the temperature exceeds a predefined threshold, the control unit 202 can adjust the feedback mechanism or initiate an automatic shutdown to ensure user safety.

The device 200 also features an integrated memory storage 222, which stores the received physiological data and feedback patterns. This allows users to log and review their data over time, providing insights into their physiological trends and history.

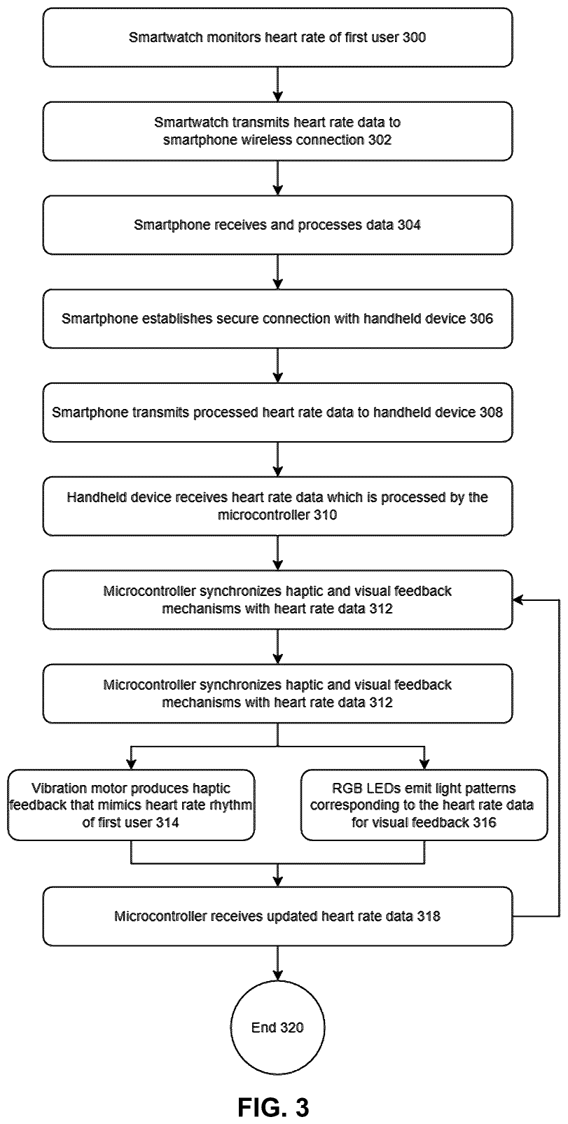

FIG. 3 illustrates a process flow diagram representing a sequence of operations carried out by the system during an example user journey. In this scenario, physiological data is transmitted from a smartwatch of a first user to a smartphone and then to the handheld device of a second user, which emulates the physiological signals.

The process begins with the smartwatch 106 monitoring the heart rate of the first user 300. In this example, only heart rate data is mentioned, however it will be understood that this could include other suitable monitored physiological data such as breathing patterns, etc. The smartwatch continuously collects heart rate data using its integrated sensors and prepares this data for transmission. The smartwatch then transmits the heart rate data to the smartphone 104 via Bluetooth Low Energy (BLE) or other suitable wireless connection 302.

Upon receiving the heart rate data, the smartphone 104 processes the data 304. This processing involves formatting the data and potentially filtering out any noise or irregularities to ensure accurate representation. The smartphone then establishes a secure connection with the handheld device 100 using a similar BLE wireless connection 306.

The smartphone transmits the processed heart rate data to the handheld device 100 over this secure BLE connection 308.

The handheld device receives the heart rate data and the control unit 202 processes the received heart rate data 310, ensuring that the data is ready for use by the feedback mechanism. The control unit then controls one or more of the haptic and visual feedback mechanisms to synchronise with the heart rate data 312.

The vibration motor 206 within the handheld device 100 is activated to produce haptic feedback that mimics the heart rate rhythm of the first user 314. Simultaneously, the RGB LEDs 208 are controlled to emit light patterns corresponding to the heart rate data, providing visual feedback 316.

The control unit then continues to receive updated heart rate data 318 and repeat the process to continuously synchronise with the first user's physiological signals in real time until the user ends the process 320.

The smartphone application enables the second user to customize the feedback settings on the handheld device. The user can adjust vibration intensity and LED color patterns according to their preferences, enhancing the personalized experience.

The handheld device 100 continues to provide synchronized haptic and visual feedback as long as it receives heart rate data from the smartphone. This real-time feedback allows the second user to experience the physiological signals of the first user, fostering a sense of emotional connectivity and awareness.

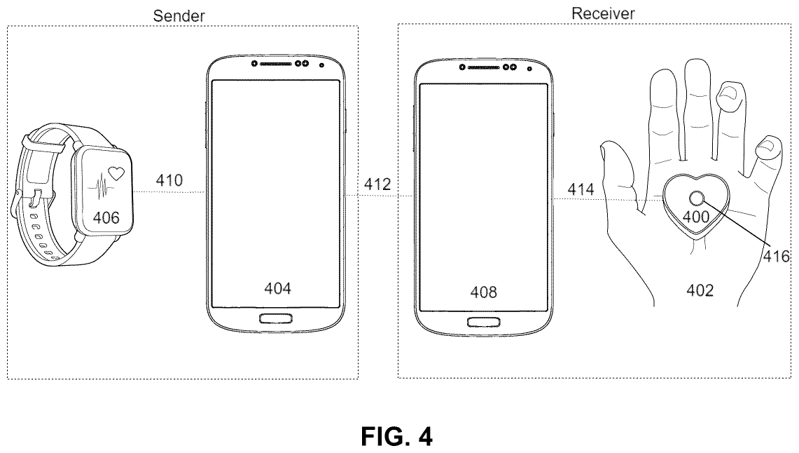

FIG. 4 illustrates an embodiment of the system architecture for real-time heart rate communication, depicting a scenario where the second user device is a second smartphone. In this alternative embodiment, the control unit of the handheld device 400 may be much simpler, requiring less circuitry for onboard processing, with said processing instead being performed by the receiver's smartphone.

The heart-shaped handheld device 400 is shown in the user's hand 402, designed to provide synchronized haptic and visual feedback based on heart rate data received from an external source. The device 400 includes a housing configured to be ergonomically held, and within this housing is a feedback mechanism and a communication module.

The system involves two main sets of devices: the sender and the receiver. The sender includes a smartwatch 406 worn by an example user, Paul, and a smartphone 404. The receiver includes Paul's mother's smartphone 408 and the handheld device 400.

Paul's smartwatch 406 continuously monitors his heart rate data using integrated sensors and transmits this data to his smartphone 404 via a Bluetooth Low Energy (BLE) connection 410. Both Paul and his mother have a dedicated application installed on their smartphones. The smartphone 404 processes the heart rate data and securely transmits it to Paul's mother's smartphone 408 via another wireless connection 412, which may be a more long range standard such as Wi-Fi, 4G, 5G, or the like.

Paul's mother receives a notification on her smartphone 408 that Paul wants to share his heart rate information. Upon accepting the request, her smartphone 408, which is paired with the handheld device 400 via BLE 414, processes the heart rate data and generates control instructions for the handheld device 400.

The feedback mechanism within the handheld device 400 includes a vibration motor and a set of RGB LEDs 416. The smartphone 408 sends control instructions to the handheld device 400 via the communication module, which interprets these instructions to actuate the feedback mechanism. The vibration motor produces haptic feedback that mimics the rhythm of Paul's heartbeat, providing a tactile representation of the heart rate data. Simultaneously, the RGB LEDs emit light patterns corresponding to the heart rate data. The smartphone application allows customization of settings such as vibration intensity and LED color patterns.

In this embodiment, the handheld device 400 is powered by a rechargeable battery. The battery management system ensures safe and efficient charging and discharging, monitored by integrated sensors that detect temperature and prevent overheating.

FIG. 4 demonstrates how the system architecture supports the transmission of heart rate data from Paul's smartwatch 406 to his smartphone 404, then to his mother's smartphone 408, and finally to the handheld device 400. This sequence allows the second user to experience Paul's heart rate in real-time through synchronized haptic and visual feedback, enhancing emotional connectivity and awareness. The handheld device continues to provide synchronized feedback as long as it receives heart rate data from Paul's mother's smartphone 408. This real-time feedback fosters a sense of emotional connection between Paul and his mother.

### Controller/Processor Components

A control unit or processor as described herein can be any suitable type of computer. A computer may be a uniprocessor or multiprocessor machine. Accordingly, a computer may include one or more processors and, thus, the aforementioned computer system may also include one or more processors. Examples of processors include sequential state machines, microprocessors, control units, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), gated logic, programmable control boards (PCBs), and other suitable hardware configured to perform the various functionality described throughout this disclosure.

Additionally, the computer may include one or more memories. Accordingly, the aforementioned computer systems may include one or more memories. A memory may include a memory storage device or an addressable storage medium which may include, by way of example, random access memory (RAM), static random access memory (SRAM), dynamic random access memory (DRAM), electronically erasable programmable read-only memory (EEPROM), programmable read-only memory (PROM), erasable programmable read-only memory (EPROM), hard disks, floppy disks, laser disk players, digital video disks, compact disks, video tapes, audio tapes, magnetic recording tracks, magnetic tunnel junction (MTJ) memory, optical memory storage, quantum mechanical storage, electronic networks, and/or other devices or technologies used to store electronic content such as programs and data. In particular, the one or more memories may store computer executable instructions that, when executed by the one or more processors, cause the one or more processors to implement the procedures and techniques described herein. The one or more processors may be operably associated with the one or more memories so that the computer executable instructions can be provided to the one or more processors for execution. For example, the one or more processors may be operably associated to the one or more memories through one or more buses. Furthermore, the computer may possess or may be operably associated with input devices (e.g., a keyboard, a keypad, controller, a mouse, a microphone, a touch screen, a sensor) and output devices such as (e.g., a computer screen, printer, or a speaker).

The computer may advantageously be equipped with a network communication device such as a network interface card, a modem, or other network connection device suitable for connecting to one or more networks.

A computer may advantageously contain control logic, or program logic, or other substrate configuration representing data and instructions, which cause the computer to operate in a specific and predefined manner as, described herein. In particular, the computer programs, when executed, enable a control processor to perform and/or cause the performance of features of the present disclosure. The control logic may advantageously be implemented as one or more modules. The modules may advantageously be configured to reside on the computer memory and execute on the one or more processors. The modules include, but are not limited to, software or hardware components that perform certain tasks. Thus, a module may include, by way of example, components, such as, software components, processes, functions, subroutines, procedures, attributes, class components, task components, object-oriented software components, segments of program code, drivers, firmware, micro code, circuitry, data, and/or the like.

The control logic conventionally includes the manipulation of digital bits by the processor and the maintenance of these bits within memory storage devices resident in one or more of the memory storage devices. Such memory storage devices may impose a physical organization upon the collection of stored data bits, which are generally stored by specific electrical or magnetic storage cells.

The control logic generally performs a sequence of computer-executed steps. These steps generally require manipulations of physical quantities. Usually, although not necessarily, these quantities take the form of electrical, magnetic, or optical signals capable of being stored, transferred, combined, compared, or otherwise manipulated. It is conventional for those skilled in the art to refer to these signals as bits, values, elements, symbols, characters, text, terms, numbers, files, or the like. It should be kept in mind, however, that these and some other terms should be associated with appropriate physical quantities for computer operations, and that these terms are merely conventional labels applied to physical quantities that exist within and during operation of the computer based on designed relationships between these physical quantities and the symbolic values they represent.

It should be understood that manipulations within the computer are often referred to in terms of adding, comparing, moving, searching, or the like, which are often associated with manual operations performed by a human operator. It is to be understood that no involvement of the human operator may be necessary, or even desirable. The operations described herein are machine operations performed in conjunction with the human operator or user that interacts with the computer or computers.

It should also be understood that the programs, modules, processes, methods, and the like, described herein are but an exemplary implementation and are not related, or limited, to any particular computer, apparatus, or computer language. Rather, various types of general-purpose computing machines or devices may be used with programs constructed in accordance with some of the teachings described herein. In some embodiments, very specific computing machines, with specific functionality, may be required.

### CONCLUSION

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the system of the invention have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A system for real-time heart rate communication, comprising:
a handheld device configured to be held by a user;
a feedback mechanism within the handheld device configured to provide sensory feedback based on received physiological data;
a control unit within the handheld device configured to interpret control instructions and actuate the feedback mechanism;
a communication module within the handheld device configured to receive the control instructions via a communication protocol and to convey the instructions to the control unit;
a first user device configured to receive physiological data from a second user device and to transmit the physiological data and control instructions to the handheld device;
wherein the first user device is configured to run an application to process the physiological data and generate control instructions for the handheld device, wherein the physiological data comprises heart rate data.

2. The system of claim 1, wherein the communication module is a Bluetooth Low Energy (BLE) module.

3. The system of claim 1, wherein the feedback mechanism comprises a vibration motor configured to provide haptic feedback.

4. The system of claim 3, wherein the feedback mechanism further comprises a plurality of RGB LEDs configured to provide visual feedback.

5. The system of claim 4, wherein the control unit is further programmed to control the RGB LEDs to emit light in synchronization with the heart rate data.

6. The system of claim 1, wherein the power source is a rechargeable battery disposed within the housing.

7. The system of claim 6, further comprising a battery management system operatively connected to the power source to manage charging and discharging of the battery.

8. The system of claim 1, wherein the feedback mechanism further comprises an audio output device configured to provide auditory feedback based on the received physiological data.

9. The system of claim 1, wherein the first user device is configured to receive physiological data from a smartwatch.

10. The system of claim 1, wherein the first user device is configured to receive physiological data from a second smartphone.

11. The system of claim 1, further comprising a secure pairing process for establishing a connection between the communication module and the first user device.

12. The system of claim 1, further comprising a user interface on the first user device application for customizing the feedback settings on the handheld device.

13. The system of claim 12, wherein the feedback settings include vibration intensity levels and RGB LED color patterns.

14. The system of claim 1, wherein the control unit is further programmed to log the physiological data and feedback patterns over time.

15. The system of claim 1, further comprising a sensor for detecting the temperature within the housing and adjusting the feedback mechanism to prevent overheating.

16. The system of claim 15, wherein the sensor triggers an automatic shutdown of the feedback mechanism upon detecting a temperature above a predefined threshold.

17. The system of claim 1, further comprising a charging port and indicator LEDs to show the battery level and charging status.

18. The system of claim 1, wherein the housing comprises a customizable exterior cover that can be replaced or modified by the user.

19. The system of claim 1, further comprising an integrated memory module for storing received physiological data and feedback patterns.

20. The system of claim 1, wherein the control unit is configured to update its firmware wirelessly through the communication module.

21. The system of claim 1, further comprising a motion sensor configured to detect the handheld device's movement and adjust the feedback mechanism accordingly.

22. The system of claim 1, wherein the physiological data includes data on the user's activity type, and the feedback mechanism adjusts its response based on this activity type.

23. The system of claim 1, further comprising a GPS module for tracking the location of the handheld device.
